(19) United States
(12) Patent Application Publication
(10) Pub. No.: US 2010/0055219 A1
(43) Pub. Date: Mar. 4, 2010

(54) ANTIINFLAMMATORY AGENT

(75) Inventors: Takeshi Yasumoto, Miyagi (JP); Hideo Naoki, Osaka (JP); Mina Hirose, Okinawa (JP); Kazuyo Tsuha, Okinawa (JP); Megumi Kuba, Okinawa (JP); Kaoru Hanashiro, Okinawa (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Tropical Technology Center Ltd., Uruma-shi (JP)

(21) Appl. No.: 12/282,457

(22) PCT Filed: Mar. 15, 2006
(86) PCT No.: PCT/JP2006/305098

§ 371 Date: Jan. 16, 2009

(51) Int. Cl. A61K 36/18

(52) U.S. Cl. 424/774

(57) ABSTRACT

An object of the invention is to find, from nature, a compound which does not have a side effect such as a hormone action or gastrointestinal disturbance as steroidal or non-steroidal anti-inflammatory agents and has an anti-inflammatory action being as good as that of those anti-inflammatory agents and is to provide an anti-inflammatory agent where an extract prepared by extraction of leaves of Yabutsubaki (Camellia japonica L.), Tsubaki (Camellia japonica L. cv.) or Sasanqua (Camellia sasanqua T.) with water, a hydrophilic solvent or a mixture thereof is an active ingredient.

### TECHNICAL FIELD

The present invention relates to an anti-inflammatory agent and, more particularly, it relates to an anti-inflammatory agent being extracted from leaves of Yabutsubaki (Camellia japonica L.), Tsubaki (Camellia japonica L. cv.) or Sasanqua (Camellia sasanqua T.) and having a degranulation inhibitory activity and a cyclooxygenase-2 inhibitory activity.

### BACKGROUND OF THE INVENTION

Drugs of steroidal and non-steroidal types have been widely used for suppression of many inflammations including allergic diseases. However, the steroidal anti-inflammatory agents have a problem of side effects such as hormone action, while the non-steroidal agents may cause clinically important enteric disorders such as gastrointestinal disorder.

As to allergic diseases continuing for a certain period of time such as pollinosis, an agent is preferred to be previously ingested as a food such as health food rather than as a drug and there has been a demand for providing an anti-inflammatory agent derived from natural substances corresponding to the above.

With regard to the component derived from natural substances having an anti-inflammatory action, it has been reported that extracts from various plants such as an extract of bark of Yamamomo (Myrica rubra) exhibit a hexosaminidase release-inhibitory activity (Non-Patent Document 1).

With regard to a cyclooxygenase-2 inhibitory activity which is one of the action mechanisms of anti-inflammatory agents, it has been reported that Tea (Camellia sinensis) and epigallocatechin 3-gallate which is a composition thereof inhibit expression of cyclooxygenase-2 and enzymatic activity thereof (Non-Patent Document 2) and, besides that, it has been also reported that an extract of Tencha (Chinese sweet tea) has a cyclooxygenase inhibitory activity (Patent Document 1).

As mentioned above, studies for anti-inflammatory action of natural substances have been continued even at present, and there has been a demand for investigating the substances derived from natural substances which have far better anti-inflammatory action. Incidentally, in the anti-inflammatory action derived from those natural substances, there has been no publication where both degranulation inhibitory activity and cyclooxygenase-2 inhibitory activity are reported.

### Patent Document 1: JP-A-9-118626

Non-Patent Document 1: Matsuda, H.; Morikawa, T.; Tao, J.; Ueda, K.; Yoshikawa, M. Chem. Pharm. Bull. (Tokyo), 2002, 50(2), 208-215
Non-Patent Document 2: Hussain, T.; Gupta, S.; Adhami, V M.; Mukhtar, H. Int. J. Cancer, 2005, 113(4), 660-669

### DISCLOSURE OF THE INVENTION

### Problems that the Invention is to Solve

Under such circumstances, there has been a brisk demand for finding compounds from nature, which unlike the steroidal and non-steroidal anti-inflammatory agents, have no side effect such as hormone action and cause no enteric disorders, and yet which exhibit equal anti-inflammatory action to the above anti-inflammatory agents. It is an objective of the present invention to provide such substances and anti-inflammatory agents utilizing them.

### Means for Solving the Problems

In order to achieve the above objective, the present inventors have intensively investigated the compounds expressing anti-inflammatory action from natural substances and found that an extract of leaves of Camellia japonica L., Camellia japonica L. cv. or Camellia sasanqua T. has a potent anti-inflammatory action whereupon the present invention has been achieved.

Thus, the present invention provides an anti-inflammatory agent in which an extract prepared by extraction of leaves of Camellia japonica L., Camellia japonica L. cv. or Camellia sasanqua T. with water, a hydrophilic solvent or a mixture thereof is an active ingredient.

### ADVANTAGES OF THE INVENTION

In the anti-inflammatory agent of the present invention, an extract prepared by extraction of leaves of Camellia japonica L., Camellia japonica L. cv. or Camellia sasanqua T. with water, a hydrophilic solvent or a mixture thereof is an active ingredient. This agent exhibits excellent degranulation inhibitory activity and cyclooxygenase-2 inhibitory activity and shows an anti-inflammatory action which is the same as or even better than the commercially available anti-inflammatory agents which have been put into the market at present.

Accordingly, the agent is able to be used either as it is or as a health food for treating or preventing various inflammations such as pollinosis, bronchial asthma, atopic dermatitis, symptoms such as pain, fever and inflammation related to influenza or other viral infections, microbe-infected pharyngitis, throat pain, bronchitis, adenoiditis, periodontitis, alveolitis, toothache, gingivitis, gout, arthritis, nephritis, hepatitis, dysmenorrhea, headache, ulcerative colitis, sprain/wrench, myalgia, neuralgia, synovitis, burn and inflammation after surgical/dental treatments.

### BEST MODES FOR CARRYING OUT THE INVENTION

An extract of leaves of Camellia japonica L., Camellia japonica L. cv. or Camellia sasanqua T. (hereinafter, it is also referred to as “an extract of camellias”) which is an active ingredient of the anti-inflammatory agent of the present invention is able to be extracted by a conventional method from leaves of Camellia japonica L., Camellia japonica L. cv. or Camellia sasanqua T.. Camellia japonica L. is a dicotyledon belonging to the family Theaceae and is a wild species which is also called Yamatsubaki. Most of horticultural varieties of camellia are differentiated from Camellia japonica L. and a lot of interspecific hybrids have been created including varieties (hereinafter, they will be referred to as “Tsubaki (Camellia japonica L. cv.)”). Camellia sasanqua T. is a dicotyledon belonging to the family Theaceae.

There is no particular limitation for the growing district and the collection period of the leaves of Camellia japonica L., Camellia japonica L. cv. or Camellia sasanqua T. used as a raw material. Although non-dried leaves may be used, dried leaves are usually used and the leaves are preferably ground or finely cut prior to an extracting operation.

As to the solvent used for extraction of leaves of Camellia japonica L., Camellia japonica L. cv. or Camellia sasanqua T., it is preferred to use water, a hydrophilic solvent or a mixture thereof. In the case of water, among them, it is preferred to use alkaline water where pH is about 8 to 12. Examples of the hydrophilic solvent include alcohols such as methanol, ethanol, propanol, isopropanol and butanol; Cellosolve; ketones such as acetone; ethers such as dioxane and tetrahydrofuran; and nitrogen-containing solvents such as pyridine, morpholine, acetonitrile, N,N-dimethylformaide, dimethylacetamide and N-methylpyrrolidone. Each of those extracting solvents may be used alone, in combination of two or more, or as a mixed solvent with water.

When the hydrophilic solvent is used as a mixed solvent with water, their ratio may be appropriately selected, for example, from the range where a ratio of water/solvent is from 95/5 to 5/95 (by volume).

Among the above-mentioned extracting solvents, examples of the particularly preferred ones are hot water and a mixed solvent of lower alcohols such as methanol and ethanol with water and more preferred one is a mixed liquid of a lower alcohol with water in which a lower alcohol is contained in such a ratio that water/solvent is from 30/70 to 70/30 (by volume).

An extraction using the above-mentioned solvent may be carried out at appropriate temperature such as from 10° C. to a refluxing temperature of the solvent or, preferably, it may be carried out at about 15 to 80° C. It is also possible to extract by means of cool percolation at room temperature. Extracting time varies depending upon extracting temperature and it is about 5 minutes to 24 hours and, preferably, from about 30 minutes to 1 hour.

The extract liquid which is extracted as above is usually concentrated to obtain a concentrated extract. If necessary, the concentrated extract may be further purified by known purifying methods. For example, water is added to the resulting concentrated extract followed by partitioning using a hydrophobic solvent whereby further purification is possible.

With regard to the hydrophobic solvent used in the above partitioning operation, there are various solvents which are able to be separated from water and examples thereof include alcohols such as butanol, isobutanol, hexanol, octanol, 2-ethylhexanol and cyclohexanol; an aromatic hydrocarbon such as benzene, toluene and xylene; a halogenated hydrocarbon such as dichloromethane, chloroform, carbon tetrachloride, dichloroethane and trichloroethylene; ethers such as ethyl ether, isopropyl ether and butyl ether; and esters such as methyl acetate, ethyl acetate and butyl acetate. Each of those hydrophobic solvents may be used alone or in combination of two or more as a mixed solvent. Among those hydrophobic solvents, butanol or the like is frequently used.

The extract liquid prepared as above may also be further purified using various kinds of columns. For example, the resulting extract liquid is passed through an adsorbent column such as HP-20, HP-21, Sepabeads SP-825, SP-850 and SP-207 (all manufactured by Mitsubishi Chemical), Sephadex LH 20 (Amersham Biosciences), Amberlite XAD4 and XAD16HP (manufactured by Rohm & Haas) and Toyopearl HW40F (manufactured by Tosoh) and separated using one or more appropriate eluant(s) to prepare a purified extract as a fraction having higher activity.

As to the solvent which is advantageously used in the above adsorbent column chromatography, there may be used, for example, water, a hydrophilic solvent such as methanol and ethanol or a mixed solvent thereof. In this step, two or more adsorption column chromatographies may be combined.

The extract of camellias prepared as above may be made into a final product as an extract in a concentrated or a non-concentrated state or as powder dried by known means such as freeze drying method. The product per se may be used as a drug. Alternatively, it may be used as an additive to food/beverage to be added to other food/beverage material for obtaining health food or common food/beverage.

Since the extract of camellias according to the present invention provides excellent degranulation inhibitory activity and cyclooxygenase-2 inhibitory activity as compared with known drugs, it also may be utilized as a degranulation inhibitor or a cyclooxygenase-2 activity inhibitor to treat diseases caused by the above activities other than anti-inflammation or to be added to food/beverage thereby preventing the diseases.

Further, the extract of camellias according to the present invention is administered to patients suffering from inflammation or being expected to have inflammation whereby the existing inflammation may be treated or the expected inflammation may be prevented. For example, when sprinkling of allergen such as pollen is expected, it is now possible to prevent the allergic inflammation by previous ingestion of the extract of camellias of the present invention.

### EXAMPLES

The present invention will now be further illustrated by way of the following Examples including test examples although the present invention is not limited by those Examples, etc. at all.

### Example 1

Preparation of an Extract of Camellia japonica L. (Using a Water-Alcohol Solvent)

Leaves (20 kg) of Camellia japonica L. (produced in Okinawa) were dried indoors for 3 to 7 days and ground using a grinder until about 3 mm width. The ground product (8.6 kg) of leaves of Camellia japonica L. was added to 70 L (about eight times by weight of the leaves) of a mixed liquid of water/methanol (3/7) and extracted by stirring overnight. After completion of the extraction, the supernatant liquid was collected and filtered with suction using filter paper to obtain an extract liquid. After the extract liquid was filtered through the filter paper, the filtrate was concentrated in vacuo until it became 21 L.

A part (about 250 mL) of the liquid concentrated in vacuo as such was subjected to a resin adsorption column chromatography where HP20 (Diaion; 250 mL) was a carrier, firstly eluted with 600 mL of a mixed liquid of water/ethanol (8/2) as a mobile phase and then eluted with 600 mL of a mixed liquid of water/ethanol (4/6), 600 mL of a mixed liquid of water/ethanol (1/9) and 500 mL of ethanol. Each of the eluted fractions was evaporated to dryness in vacuo to obtain an extract. Yields of the extracts were 7.3 g for a water/ethanol (8/2) fraction, 1.86 g for a water/ethanol (4/6) fraction, 91.9 mg for a water/ethanol (1/9) fraction and 8.8 mg for an ethanol fraction.

### Example 2

Preparation of Extracts of Camellia japonica L. and Kantsubaki (Extraction with Hot Water)

(1) Leaves (about 2 g) of Camellia japonica L. (produced in Okinawa) were dried, then dried at 60° C. for 2 hours and ground using a mixer. The resulting ground product of leaves of Camellia japonica L. was added to 50 mL of hot water (about 95 to 100° C.) and extracted for 30 minutes. After extracting with hot water, the extract was cooled down to about 40° C. and filtered through filter paper to obtain an extract liquid.

(2) Leaves of Kantsubaki (Camellia hiemalis ‘Shishigashira’, a horticultural hybrid of Camellia sasanqua T.) (produced in Miyagi) were dried indoors and ground to an extent of about 3 to 5 mm width using a grinder. The resulting ground leaves (100 g) were added to hot water (about 95 to 100° C.) followed by extracting for 30 minutes. After the extraction with hot water, the extract was cooled down to about 40° C. and filtered through filter paper to give an extract liquid.

### Example 3

Preparation of an Extract of Camellia japonica L. (Extraction with Alkali)

Leaves (2.6 g) of Camellia japonica L. were dried at 60° C. for 2 hours and roughly cut to an extent of about 6 mm using scissors. To this was added 50 mL of a 0.01N aqueous solution of sodium hydroxide, the mixture was ground for 2 minutes using a homogenizer and centrifuged at 3,000 rpm for 5 minutes at 4° C. and the supernatant thus obtained was collected. 50 mL of a 0.01N aqueous solution of sodium hydroxide was freshly added to the residue, and the same operation was repeated twice. The supernatants thus obtained were combined, filtered, adjusted to pH 7.0 and evaporated to dryness in vacuo to give an extract.

### Example 4

Preparation of an Extract of Camellia japonica L. cv. (Extraction under Refluxing)

Raw leaves (about 50 g) of Camellia japonica L. cv. were roughly cut to an extent of about 6 mm using scissors, dipped in 0.5 L of a mixed liquid of water/methanol (2/8) and extracted for 30 minutes at a refluxing temperature of the solvent. The supernatant thus obtained was filtered and evaporated to dryness in vacuo to give an extract.

### Example 5

Preparation of Extracts of Camellias from Various Production Areas

Each 1 to 2 g of leaves of Camellia japonica L., Camellia japonica L. cv. or Camellia sasanqua T. produced in various areas and horticultural species thereof was obtained and dried at 60° C. for 2 hours and roughly cut to an extent of about 6 mm using scissors. 50 mL of a mixed liquid of water/ethanol (3/7) was added to the leaves, and grinding and extraction by stirring were carried out using a homogenizer for 2 minutes. After that, the extract was centrifuged at 3,000 rpm for 5 minutes at 4° C. and the supernatant thus obtained was collected.

To the residue was freshly added 50 mL of a mixed liquid of water/ethanol (3/7) and the same operation was repeated twice. The supernatant thus obtained were combined, filtered and evaporated to dryness in vacuo to give an extract.

### Example 6

Measurement of Degranulation Inhibitory Activity:

With regard to the measurement of degranulation inhibitory activity, a test for hexosaminidase release-inhibitory activity was carried out by referring to Non-Patent Document 1 and Non-Patent Document 3 (Kataoka M., Takagaki Y., Shoyakugaku Zasshi, 46(1), 25-29, 1992). Firstly, rat basophilic leukemia cells (RBL-2H3) were made into 5×10⁵ cells/mL and seeded on a 96-well plate and anti-DNP-BSA mouse IgE antibody was added thereto so as to make its final concentration 0.29 μg/mL and incubated with 5% CO₂ at 37° C. overnight in an incubator to sensitize the cells. Then the cells were washed with a phosphate-buffered physiological saline solution twice and 130 μL of a releasing mixture (comprising 116.9 mM of NaCl, 5.4 mM of KCl, 0.8 mM of MgSO₄, 2.0 mM of CaCl₂, 5.6 mM of glucose, 0.1% of bovine serum albumin and 25 mM of HEPES) was added thereto.

After that, each extract and fraction (10 μL) of the present invention (prepared by firstly dissolving in 50% ethanol followed by dissolving with 1% ethanol so as to make the final concentration 10 μg/mL) was prepared to give concentrations of five to six stages and allowed to stand for 10 minutes with 5% of CO₂ at 37° C. in an incubator. Then 10 μL of an antigen DNP-BSA (2 μg/mL) was added, the mixture was allowed to stand in an incubator for 1 hour to induce degranulation, and centrifuged to collect the supernatant. 15 μL of a 5 mM hexosaminidase substrate solution (p-nitrophenyl-β-D-glucosaminide) was added to 45 μL of the supernatant liquid, the mixture was made to react at 37° C. for 3 hours and 180 μL of a solution for stopping the reaction (0.1M NaHCO₃/Na₂CO₃; pH 10.0) was added thereto. After completion of the reaction, absorbance at 415 nm was measured and the hexosaminidase release-inhibitory activity was calculated by the following formula. Results for Camellia japonica L. are shown in Table 1 while results for all others are shown in Table 2. Meanwhile, a positive control (200 μM of ketotifen fumarate) and a negative control corresponding to the final solvent concentration for the test substance were prepared.

Hexosaminidase release-inhibitory Activity(%)=[1−(S−B/C−b)]×100

S: absorbance of the test substance upon addition of cells

B: absorbance upon addition of the test substance in the absence of the cells

C: absorbance of the negative control

b: absorbance in the absence of cells

TABLE 1

| | | | Degranulation Inhibitory Activity |
|---|---|---|---|
| Samples Used ^{Note)} | | | (IC₅₀) (μg/mL) |
| Camellia japonica L. | Wakayama | Dried Leaves | 5.32 |
| Extract with water/ethanol (3/7) | | | |
| Camellia japonica L. | Hiroshima | Dried Leaves | 2.58 |
| Extract with water/ethanol (3/7) | | | |
| Camellia japonica L. | Okinawa [Nakijin] | Crude Leaves | 7.70 |
| Extract with water/ethanol (3/7) | | | |
| Camellia japonica L. | Okinawa [Nakijin] | Dried Leaves | 2.37 |
| Extract with water/ethanol (3/7) | | | |
| Camellia japonica L. | Okinawa [Nakijin] | Dried Leaves | 4.98 |
| Extract with water/ethanol (5/5) | | | |
| Camellia japonica L. | Okinawa [Nakijin] | Dried Leaves | 5.98 |
| Extract with water/ethanol (7/3) | | | |
| Camellia japonica L. | Okinawa [Nakijin] | Dried Leaves | 43.73 |
| Extract with 0.01N NaOH | | | |
| Camellia japonica L. | Okinawa [Nago] | Dried Leaves | 9.41 |
| Extract with hot water | | | |
| Camellia japonica L. | Okinawa [Nago] | Dried Leaves | 7.75 |
| Extract with water/methanol (3/7) | | | |
| Camellia japonica L. | Okinawa [Nago] | Dried Leaves | 55.06 |
| HP20 water/ethanol (8/2) elution fraction | | | |
| Camellia japonica L. | Okinawa [Nago] | Dried Leaves | 5.38 |
| HP20 water/ethanol (4/6) elution fraction | | | |
| Camellia japonica L. | Okinawa [Nago] | Dried Leaves | 5.34 |
| HP20 water/ethanol (1/9) elution fraction | | | |
| Camellia japonica L. | Okinawa [Okinawa] | Crude leaves | 7.05 |
| Extract with water/ethanol (3/7) | | | |
| Camellia japonica L. | Okinawa [Okinawa] | Dried leaves | 7.19 |
| Extract with water/ethanol (3/7) | | | |
| Ketotifen fumarate | | | 71.75 |

^{Note)} Each sample is shown in the order of plant name; produced place (name of the prefecture); material used; and extracting method. With regard to a fraction eluted with HP20 water/ethanol however, it means a fractionated fraction of the extract with water/methanol (3/7) using HP20.

TABLE 2

| | | | Degranulation Inhibitory Activity |
|---|---|---|---|
| Samples Used ^{Note)} | | | (IC₅₀) (μg/mL) |
| Camellia japonica L. cv. | Tokyo | Crude leaves | 2.83 |
| Extract with water/methanol (2/8) under refluxing | | | |
| Camellia japonica L. cv. | Tokyo | Dried leaves | 3.78 |
| Extract with water/ethanol (3/7) | | | |
| Camellia japonica L. cv. | Osaka | Dried leaves | 2.68 |
| Extract with water/ethanol (3/7) | | | |
| Camellia japonica L. cv. | Kagawa | Dried leaves | 3.02 |
| Extract with water/ethanol (3/7) | | | |
| Camellia japonica L. cv. | Okinawa | Dried leaves | 4.68 |
| Extract with water/ethanol (3/7) | | | |
| Kantsubaki | Miyagi | Indoor-dried leaves | 3.26 |
| Extract with water/ethanol (3/7) | | | |
| Kantsubaki | Miyagi | Sun-dried leaves | 11.97 |
| Extract with water/ethanol (3/7) | | | |
| Kantsubaki | Miyagi | Indoor-dried leaves | 10.95 |
| Extract with hot water | | | |
| Kantsubaki | Hiroshima | Dried leaves | 16.75 |
| Extract with water/ethanol (3/7) | | | |
| Kantsubaki | Okinawa | Dried leaves | 2.99 |
| Extract with water/ethanol (3/7) | | | |
| Ketotifen fumarate | | | 71.75 |

^{Note)} Each sample is shown in the order of plant name; produced place (name of the prefecture); material used; and extracting method.
* Kantsubaki is a horticultural species of Camellia sasanqua T.

As the result, the IC₅₀ value of an extract of Camellia japonica L. (produced in Okinawa) of Example 1 of the present invention in terms of inhibitory activity of an extract with a mixed liquid of water/methanol (3/7) was 7.75 μg/mL. After fractionation with HP20, it was 55.06 μg/mL for a water-ethanol (8/2) fraction, 5.38 μg/mL for a water-ethanol (4/6) fraction and 5.34 μg/mL for a water-ethanol (1/9) fraction. Among the above, the water-ethanol (4/6) fraction and water-ethanol (1/9) fraction showed nearly the same high activity. When the yield of Example 1 is taken into consideration, the water/ethanol (4/6) fraction is preferred since it showed high activity and high yield. In an extract with hot water, it was 9.41 μg/mL for Camellia japonica L. cv. and 10.95 μg/mL for Kantsubaki while it was 71.75 μg/mL for ketotifen fumarate which is a positive control, whereby all of the extracts of the present invention and fractions prepared therefrom showed higher activity than ketotifen fumarate.

### Example 7

Measurement of Cyclooxygenase-2 Inhibitory Activity:

A cyclooxygenase-2 inhibitory activity (COX-2 inhibitory activity) was measured by an enzyme immunoassay method using a COX inhibitor screening assay kit (Cayman Chemical) as follows. Thus, 970 μL of 0.1M Tris hydrochloride buffer (containing 5 mM of ethylenediamine tetraacetate and 2 mM of phenol; pH 8.0), 10 μL of hem solution, 10 μL of cyclooxygenase-2 (recombinant product derived from human) solution and 20 μL of sample solution were mixed in a microtube and pre-incubated at 37° C. for 10 minutes. To this was added 10 μL of arachidonic acid solution followed by being made to react at 37° C. for 2 minutes. The reaction was stopped by addition of 50 μL of 0.2M hydrochloric acid, then 100 μL of 0.2M stannous chloride solution was added thereto and the mixture was allowed to stand at room temperature for 5 minutes. Meanwhile, there were also prepared a blank where the same operation was carried out after the enzyme was previously inactivated and a control where the enzymatic reaction was not inhibited due to non-addition of a sample.

An enzymatic reaction solution (50 μL), 50 μL of a prostaglandin screening tracer and 50 μL of a prostaglandin screening antiserum were added to a 96-well plate for an enzyme immunoassay attached to the kit and made to react at room temperature for 18 hours. After washing it with a washing buffer five times, 200 μL of Ellman's reagent was added, and incubation was carried out at room temperature for 30 minutes. After the incubation, absorbance at 405 nm was measured and prostaglandin F₂α which is a reaction product was quantified. Cyclooxygenase-2 inhibitory activity was expressed in terms of an inhibiting rate calculated by the following formula.

Cyclooxygenase-2 inhibiting rate (%)=[(A−B)−(C−B)]/(A−B)×100

A: produced amount of prostaglandin F₂α in the control

B: produced amount of prostaglandin F₂α in the blank

C: produced amount of prostaglandin F₂α upon addition of a sample

The cyclooxygenase-2 inhibitory activity for extracts of Camellia japonica L. from various production places and fractionated fractions thereof obtained in Examples 1 to 5 is shown in Table 3 and that for extracts of Camellia japonica L. cv. and Camellia sasanqua T. is shown in Table 4. The result was that a cyclooxygenase-2 inhibitory activity was noted in all of the extracts of Camellia japonica L., Camellia japonica L. cv., Camellia sasanqua T. and the horticultural species thereof.

TABLE 3

| | | | Sample | COX-2 |
|---|---|---|---|---|
| | | | Concentration | Inhibiting Rate |
| Samples Used ^{Note)} | | | (mg/mL) | (%) |
| Camellia japonica L. | Wakayama | Dried Leaves | 0.1 | 42.8 |
| Extract with water/ethanol (3/7) | | | 0.5 | 77.9 |
| | | | 1.0 | 94.4 |
| Camellia japonica L. | Hiroshima | Dried Leaves | 0.1 | 36.3 |
| Extract with water/ethanol (3/7) | | | 0.5 | 94.7 |
| | | | 1.0 | 95.0 |
| Camellia japonica L. | Okinawa [Nakijin] | Crude leaves | 0.1 | 65.7 |
| Extract with water/ethanol (3/7) | | | 0.5 | 91.9 |
| | | | 1.0 | 97.0 |
| Camellia japonica L. | Okinawa [Nakijin] | Dried Leaves | 0.5 | 47.2 |
| Extract with water/ethanol (5/5) | | | 1.0 | 73.0 |
| Camellia japonica L. | Okinawa [Nakijin] | Dried Leaves | 0.5 | 34.9 |
| Extract with water/ethanol (7/3) | | | 1.0 | 65.7 |
| Camellia japonica L. | Okinawa [Nago] | Dried Leaves | 0.1 | 91.6 |
| Extract with water/ethanol (3/7) | | | 0.5 | 97.6 |
| | | | 1.0 | 95.9 |
| Camellia japonica L. | Okinawa [Nago] | Dried Leaves | 0.3 | 73.9 |
| HP20 water/ethanol (8/2) elution fraction | | | | |
| Camellia japonica L. | Okinawa [Nago] | Dried Leaves | 0.1 | 33.6 |
| HP20 water/ethanol (4/6) elution fraction | | | 0.3 | 83.8 |
| Indomethacin | | | 1 μM | 87.1 |

^{Note)} Each sample is shown in the order of plant name; produced place (name of the prefecture); material used; and extracting method. With regard to a fraction eluted with HP20 water/ethanol however, it means a fractionated fraction of the extract with water/methanol (3/7) using HP20.

TABLE 4

| | | | Sample | COX-2 |
|---|---|---|---|---|
| | | | Concentration | Inhibiting Rate |
| Samples Used ^{Note)} | | | (mg/ml) | (%) |
| Camellia japonica L. cv. | Tokyo | Crude leaves | 0.1 | 40.4 |
| Extract with water/methanol (2/8) under refluxing | | | 0.5 | 77.9 |
| | | | 1.0 | 90.1 |
| Camellia japonica L. cv. | Osaka | Dried leaves | 0.1 | 50.4 |
| Extract with water/ethanol (3/7) | | | 0.5 | 80.9 |
| | | | 1.0 | 89.4 |
| Camellia japonica L. cv. | Kagawa | Dried leaves | 0.5 | 88.0 |
| Extract with water/ethanol (3/7) | | | 1.0 | 96.1 |
| Camellia japonica L. cv. | Okinawa | Dried leaves | 0.1 | 36.7 |
| Extract with water/ethanol (3/7) | | | 0.5 | 86.6 |
| | | | 1.0 | 94.5 |
| Kantsubaki | Miyagi | Indoor-dried leaves | 0.1 | 49.2 |
| Extract with water/ethanol (3/7) | | | 0.5 | 96.4 |
| | | | 1.0 | 97.1 |
| Kantsubaki | Miyagi | Indoor-dried leaves | 0.5 | 62.8 |
| Extract with hot water | | | 1.0 | 81.3 |
| Kantsubaki | Hiroshima | Dried leaves | 0.1 | 59.1 |
| Extract with water/ethanol (3/7) | | | 0.5 | 94.2 |
| | | | 1.0 | 96.1 |
| Kantsubaki | Okinawa | Dried leaves | 1.0 | 58.5 |
| Extract with water/ethanol (3/7) | | | | |
| Camellia sasanqua T. | Okinawa | Dried leaves | 0.1 | 67.0 |
| Extract with water/ethanol (3/7) | | | 0.5 | 95.4 |
| | | | 1.0 | 96.6 |
| Indomethacin | | | 1 μM | 87.1 |

^{Note)} Each sample is shown in the order of plant name; produced place (name of the prefecture); material used; and extracting method.
* Kantsubaki is a horticultural species of Camellia sasanqua T.

### INDUSTRIAL APPLICABILITY

In the allergic reaction causing inflammation, there are generally four types such as anaphylaxis (type I), cytotoxic type (type II), Arthus type (type III) and cell-mediated type (delayed type) (type IV). Pollinosis which has been particularly becoming a problem in recent years is classified under the type I allergy (immediate type allergy). Although it has been said that atopic dermatitis mainly comprises the type I allergic reaction as well, it has been found recently that the type IV allergic reaction also participates in that.

Reaction mechanism of this type I (immediate type) allergy is that IgE produced by B cells is bonded to a highly affinitive IgE receptor existing on cell membrane of basophiles or mast cells and exogenous antigen cross-links to IgE on cell membrane whereupon a mediator such as histamine or leukotriene is released to result in onset of allergy. Hexosaminidase is an enzyme released from leukocytes and has been known to correlate with the histamine release. Therefore, in order to prevent the type I allergic reaction, any of the above pathways is to be cut.

One of the action mechanisms of non-steroidal anti-inflammatory agents is a cyclooxygenase inhibitory action. Cyclooxygenase catalyzes the reaction of the production of prostaglandins from arachidonic acid. Prostaglandins exhibit various physiological activities such as bronchial contraction, uterine contraction and platelet aggregation. In addition, they also induce and promote the inflammation reaction in various parts of living body.

As shown in each of the above Examples, an extract of leaves of Camellia japonica L., Camellia japonica L. cv. or Camellia sasanqua T. has excellent degranulation inhibitory activity and cyclooxygenase-2 inhibitory activity and, therefore, the extract is very effective for the treatment and prevention of diseases caused by inflammation.

Consequently, the extract of Camellia japonica L., Camellia japonica L. cv. or Camellia sasanqua T. of the present invention may be used as an anti-inflammatory agent or an anti-inflammatory ingredient for drugs for human and animals, a material for drugs, or a food material.

1-11. (canceled)

12. An anti-inflammatory agent, comprising, as an active ingredient, an extract prepared by the extraction of leaves of Yabutsubaki (Camellia japonica L.), Tsubaki (Camellia japonica L. cv.) or Sasanqua (Camellia sasanqua T. ) with water or a mixture of water with a hydrophilic solvent.

13. The anti-inflammatory agent according to claim 12, wherein the water used for the extraction is alkaline water.

14. The anti-inflammatory agent according to claim 12, wherein the hydrophilic solvent used for the extraction is a lower alcohol.

15. The anti-inflammatory agent according to claim 12, wherein the agent has a degranulation inhibitory activity and a cyclooxygenase-2 inhibitory activity.

16. The anti-inflammatory agent according to claim 12, wherein the agent is a drug.

17. The anti-inflammatory agent according to claim 12, wherein the agent is an additive to a food/beverage.

18. A food/beverage, comprising an extract added to a food material, wherein the extract is prepared by the extraction of leaves of Yabutsubaki (Camellia japonica L.), Tsubaki (Camellia japonica L. cv.) or Sasanqua (Camellia sasanqua T.) with water or a mixture of water with a hydrophilic solvent.

19. A degranulation inhibitor comprising, as an active ingredient, an extract prepared by the extraction of leaves of Yabutsubaki (Camellia japonica L.), Tsubaki (Camellia japonica L. cv.) or Sasanqua (Camellia sasanqua T.) with water or a mixture of water with a hydrophilic solvent.

20. A cyclooxygenase-2 inhibitor comprising, as an active ingredient, an extract prepared by the extraction of leaves of Yabutsubaki (Camellia japonica L.), Tsubaki (Camellia japonica L. cv.) or Sasanqua (Camellia sasanqua T. ) with water or a mixture of water with a hydrophilic solvent.

21. A method for the manufacture of an anti-inflammatory agent comprising incorporating into a carrier an extract prepared by the extraction of leaves of Yabutsubaki (Camellia japonica L.), Tsubaki (Camellia japonica L. cv.) or Sasanqua (Camellia sasanqua T.) with water or a mixture of water with a hydrophilic solvent for the manufacture of an anti-inflammatory agent.
